(19) United States
(12) Patent Application Publication
(10) Pub. No.: US 2008/0009811 A1
(43) Pub. Date: Jan. 10, 2008

(54) Non-Skin-Contacting Microneedle Array Applicator

(75) Inventor: Adam S. Cantor, River Falls, WI (US)

Correspondence Address: 3M INNOVATIVE PROPERTIES COMPANY, PO BOX 33427, ST. PAUL, MN, 55133-3427, US

(73) Assignee: 3M INNOVATIVE PROPERTIES COMPANY, Saint Paul, MN (US)

(21) Appl. No.: 11/718,444

(22) PCT Filed: Nov. 18, 2005
(86) PCT No.: PCT/US05/41806

§ 371 Date: May 2, 2007

Related U.S. Application Data
(60) Provisional application No. 60/629,186, filed on Nov. 18, 2004.

(51) Int. Cl. A61M 5/158

(52) U.S. Cl. 604/272

(57) ABSTRACT

An applicator used to apply microneedle arrays to a mammal. An applicator capable of sensing a controlled distance from a skin surface and propelling a microneedle array across this distance and into the skin surface is disclosed. A method of applying a microneedle array to a skin surface by placing the microneedle array a predetermined distance away from the skin surface and propelling the microneedle array into the skin surface is disclosed.

### CROSS-REFERENCE TO RELATED APPLICATIONS

The present application claims priority to U.S. Provisional Application Serial No. 60/629,186, filed on Nov. 18, 2004, which is incorporated herein in its entirety.

### FIELD

The present invention relates to applicators used to apply microneedle arrays to a mammal. The present method also relates to methods of applying a microneedle array or patch to a mammal.

### BACKGROUND

Only a limited number of molecules with demonstrated therapeutic value can be transported through the skin, even with the use of approved chemical enhancers. The main barrier to transport of molecules through the skin is the stratum comeum (the outermost layer of the skin).

Devices including arrays of relatively small structures, sometimes referred to as microneedles or micro-pins, have been disclosed for use in connection with the delivery of therapeutic agents and other substances through the skin and other surfaces. The devices are typically pressed against the skin in an effort to pierce the stratum corneum such that the therapeutic agents and other substances can pass through that layer and into the tissues below.

Issues related to applying microneedles include the ability to effectively insert the needles to a desired depth in the skin and the ability to protect the delicate microneedles prior to application to the skin. A number of different applicators have been proposed for use in applying microneedle arrays, but a common feature to all of these applicators is the need to place the applicator in contact with a skin surface in order to apply the microneedle array.

### SUMMARY OF THE INVENTION

The present invention provides a method of applying a microneedle array to a skin surface without the applicator contacting the skin surface.

In one embodiment, the present invention provides an applicator capable of sensing a controlled distance from a skin surface and propelling a microneedle array across this distance and into the skin surface.

In another embodiment, the present invention provides a method of applying a microneedle array to a skin surface by placing the microneedle array a predetermined distance away from the skin surface and propelling the microneedle array into the skin surface.

In another embodiment, the present invention provides an application device for applying a microneedle device to a skin surface comprising a means for releasably retaining a microneedle array, means for remotely detecting a distance between the applicator and a skin surface, means for allowing triggering of the applicator in response to the detected distance, and means for propelling the microneedle array into a skin surface.

In another embodiment, the present invention provides an application device for applying a microneedle device to a skin surface comprising a means for releasably retaining a microneedle array, a distance sensor capable of remotely sensing distance, a triggering mechanism controlled by the distance sensor, and means for propelling the microneedle array into a skin surface.

As used herein, certain terms will be understood to have the meaning set forth below:

“Array” refers to the medical devices described herein that include one or more structures capable of piercing the stratum corneum to facilitate the transdermal delivery of therapeutic agents or the sampling of fluids through or to the skin.

“Microstructure,” “microneedle” or “microarray” refers to the specific microscopic structures associated with the array that are capable of piercing the stratum corneum to facilitate the transdermal delivery of therapeutic agents or the sampling of fluids through the skin. By way of example, microstructures can include needle or needle-like structures as well as other structures capable of piercing the stratum corneum.

The features and advantages of the present invention will be understood upon consideration of the detailed description of the preferred embodiment as well as the appended claims. These and other features and advantages of the invention may be described below in connection with various illustrative embodiments of the invention. The above summary of the present invention is not intended to describe each disclosed embodiment or every implementation of the present invention. The Figures and the detailed description which follow more particularly exemplify illustrative embodiments.

### BRIEF DESCRIPTION OF THE DRAWINGS

Preferred embodiments of the invention will now be described in greater detail below with reference to the attached drawings, wherein:

FIG. 1A is a schematic cross-sectional view of one embodiment of the microneedle array application device of the present invention.

FIG. 1B is a schematic cross-sectional view of a microneedle array applied to a skin surface by the application device of FIG. 1A.

FIG. 2 is a schematic perspective view of a patch microneedle device.

FIG. 3 is a schematic cross-sectional view of another embodiment of the microneedle array application device of the present invention.

FIG. 4 is a schematic cross-sectional view of a portion of the device of FIG. 3 shown in a deployed state.

While the above-identified drawing figures set forth several embodiments of the invention, other embodiments are also contemplated, as noted in the discussion. In all cases, this disclosure presents the invention by way of representation and not limitation. It should be understood that numerous other modifications and embodiments can be devised by those skilled in the art, which fall within the scope and spirit of the principles of the invention. The figures may not be drawn to scale. Like reference numbers may have been used throughout the figures to denote like parts.

### DETAILED DESCRIPTION

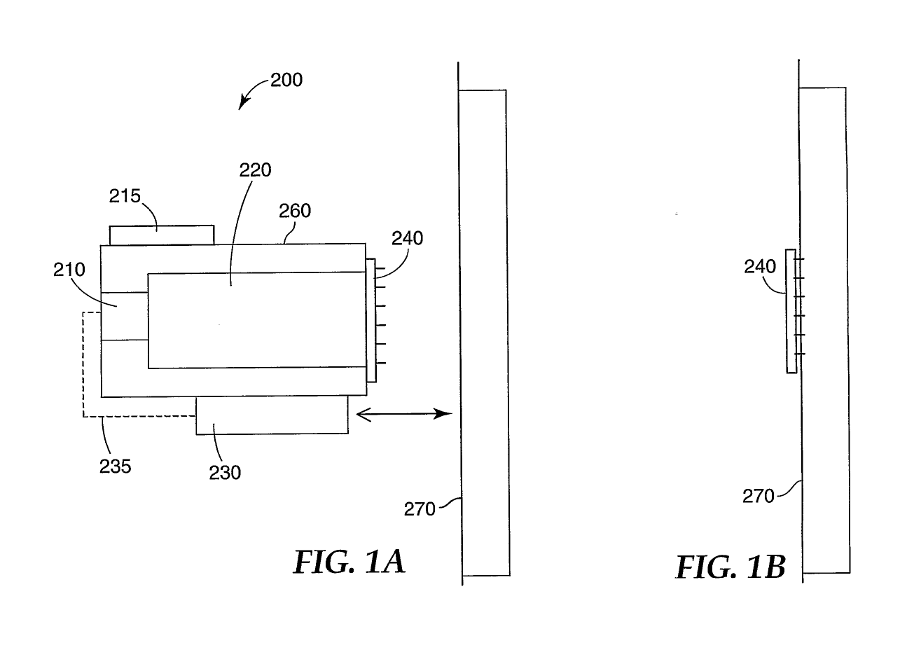

One embodiment of the microneedle application device is shown schematically in FIG. 1A. The application device 200 comprises a housing 260 that houses a piston 220 and a trigger release mechanism 210. The microneedle array 240 is releasably attached to the housing 260 by attachment means (not shown). A distance sensor 230 is connected to the outer part of the housing 260. The distance sensor is any suitable sensor capable of measuring the distance between the sensor and a remote object, such as a skin surface 270. The sensor 230 communicates with the trigger release 210 by a controller 235.

In operation, the trigger 215 is incapable of firing the piston 220 unless the trigger release mechanism 210 is receiving input from the sensor 230 indicating that the sensor 230 is within a predetermined range of distance from a target surface. Thus, the trigger release mechanism 210 serves as both a safety mechanism and a positioning mechanism to prevent the trigger 215 from firing the piston 220 unless the device is placed at a desired distance from a target surface, preferably a skin surface. If the trigger 215 is pressed or otherwise activated when the sensor 230 indicates that the device is either too far from or too close to the skin surface, then the piston 220 will not fire. In practice, the trigger may be activated when the application device is still too far from the skin surface and when the device is brought to within the predetermined distance the piston will fire.

The sensor 230 may be any conventional distance sensor, such as a photo-reflective sensor, a laser triangulation sensor, or an ultrasonic sensor. Typical sensors have a transmitter that emits a signal, such as a light wave or sound wave, and a receiver that receives a reflection of the emitted signal from a target surface. The distance between sensor and target surface may be determined by analyzing the intensity of the reflected signal, the angular distribution of the reflected signal, and/or the time it takes for the reflected signal to return to the sensor. The sensor is in a fixed relationship to the microneedle array that is releasably attached to the application device, so that a measurement of the distance of the sensor from the target surface may be easily converted into the distance between the microneedle array and the target surface.

In one embodiment, the sensor desirably indicates direction of movement and/or angular positioning of the microneedle array with respect to the target surface. The controller may be desirably configured so as to allow triggering only when the microneedle array is positioned at an appropriate distance and angular position from a skin surface and such that the relative rate of motion of the array with respect to the target surface is below a predetermined threshold value. That is, the microneedle array is desirably positioned at a fixed distance and orientation in a relatively motionless position with respect to the skin surface before the trigger release mechanism is activated.

The controller 235 may communicate with the trigger release mechanism 210 by any suitable electrical or mechanical means. The trigger release mechanism 210 may be for instance a solenoid that is activated by the controller to release a latch or pin that will prevent the piston from firing when the latch or pin is in a non-released position.

The releasable attachment means for connecting the microneedle array to the housing and/or piston may be any type of suitable means known to one skilled in the art, such as a repositionable adhesive, hook and loop connection, magnetic connection, mechanical interference fit, or snap-fit connection. The piston operation may also be any suitable type of piston design known to one skilled in the art. Further description of releasable attachment means and piston designs are described in U.S. Pat. No. 6,293,925 and U.S. Patent Application Publications 2002/0091357, 2002/0123675, 2002/0087182 and U.S. Patent Application Ser. No. 60/578651, the disclosures of which are herein incorporated by reference.

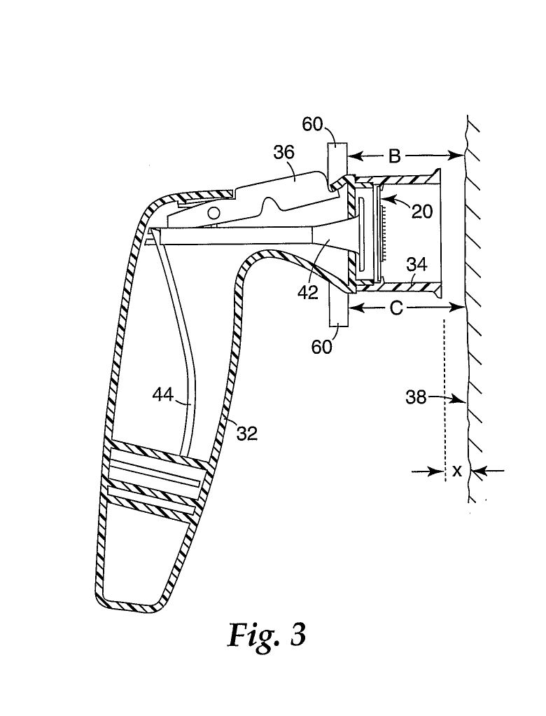

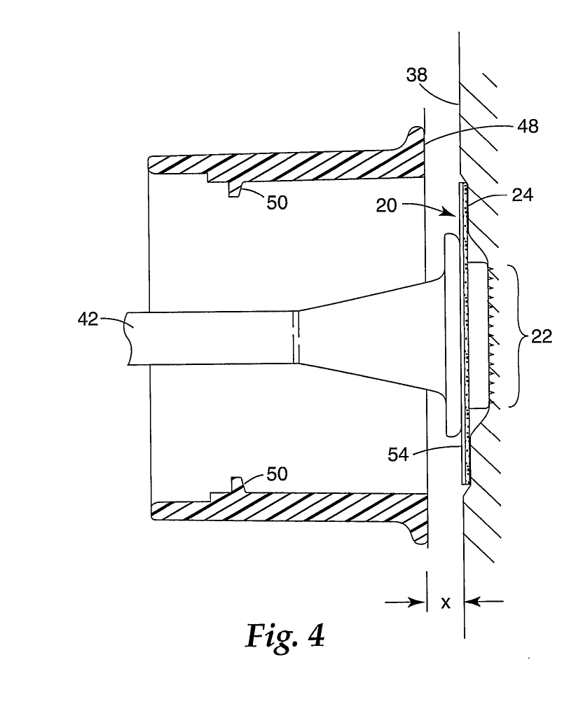

In one embodiment, the piston may extend beyond the housing to press the microneedle array into the skin surface. The array contacts the skin, whereas the piston is held away from the skin surface by the array. This is shown in more detail in FIGS. 3 and 4. A microneedle array application device is shown in FIG. 3 in a first stored position where the actuator 36 has not been engaged. The driver 44 has stored energy and the piston 42 is not in contact with the patch 20, which is retained within the collar 34 of the application device. The application device has distance sensors 60 that sense distances ‘B’ and ° C.′ between the sensor and a skin surface. The user may bring the applicator in proximity to a skin surface 38 so that the distances ‘B’ and ° C.40 may be adjusted so that the distance, x, between the end of the collar 34 and the skin surface will be as desired. Once the distance and orientation of the application device is as desired (e.g., when B and C are equivalent and the distance x is less than the distance that the piston 42 protrudes from the device after activation), then the application device is triggered. A portion of the application device is shown in FIG. 4 in the second released or triggered position, where the actuator 36 has been engaged, allowing the driver 44 to urge the piston 42 towards the patch 20, thereby removing the patch from the holding tabs 50, propelling the patch 20 beyond the open distal end 48 of the collar 34 and pressing the microneedle array 22 and a skin facing adhesive 24 against the skin 38. The piston 42 may then be removed from contact with the patch 20, thereby leaving the patch 20 in place on the skin 38. In an alternative embodiment, the piston 42 may propel the patch 20 and array 22 from the application device and the patch 20 and array 22 may travel part of the distance in air (not shown) before impacting with the skin surface 38. The microneedle array applied to a skin surface 270 with the application device having been removed, is shown in FIG. 1B.

In alternative embodiments, the means for propelling the microneedle array into the skin surface may be selected from other energy sources, such as pressure, electricity, elastic bands, and magnets.

In another embodiment the application device may be configured with two or more sensors positioned around the housing such that the orientation of the microneedle array has to be generally parallel to the skin surface prior to the trigger release being activated.

In another embodiment the trigger may be automatically activated when the trigger release is activated. In such an embodiment it may be desired to have a cocking or arming mechanism that must be activated before the automatic trigger release is enabled. The application device may be configured so that it is reloadable, that is, so that it may be used repeated times for applying microneedle arrays.

It should be understood that the application device may make insubstantial or inconsequential contact with the skin surface and still be generally considered a non-skin-contacting device. For example, a user may cause incidental contact of the applicator with a skin surface during the process of properly aligning the applicator. In one embodiment, it may be desired to bring the applicator just to the point of contact with a skin surface without actually resting or pressing the applicator against the skin. In such an instance, the applicator may gently brush the skin in an inconsequential fashion. In another embodiment, the skin facing surface of the applicator may be equipped with one or more thin, flexible fibers that can be used to provide a visual indicator to the user regarding the approximate distance at which the applicator should be placed for proper deployment of the microneedle array. In such an instance, the end of one or more of the fibers may gently brush the skin in an inconsequential fashion.

A method of applying a microneedle device using an application device of the present invention involves having the microneedle device reach a desired velocity that is effective to pierce the microneedles into the skin. The desired velocity is controlled to limit or prevent stimulation of the underlying nerve tissue. In connection with the present invention, the maximum velocity achieved by the microneedle device upon impact with the skin is often 20 meters per second (m/s) or less, potentially 15 m/s or less, and possibly 10 m/s or less. In some instances, the maximum velocity be 8 m/s or less. At the lower end of the range of velocities, the minimum velocity achieved by the microneedle device upon impact with the skin is often 2 m/s or more, potentially 4 m/s or more, and possibly 6 m/s or more.

The force required to reach the desired velocities may vary based on the mass and shape of the microneedle application device. The mass of the microneedle application device may be controlled or selected to reduce the likelihood that nerve tissue underneath the delivery site is stimulated sufficiently to result in the sensation of pain.

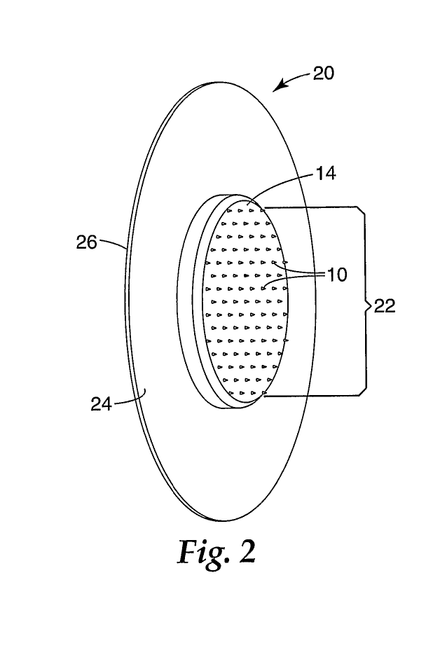

In one embodiment, the microneedle device shown schematically as 240 in FIGS. 1A, B may be in the form of a patch shown in more detail in FIG. 2. FIG. 2 illustrates a microneedle device comprising a patch 20 in the form of a combination of an array 22, pressure sensitive adhesive 24 and backing 26. A portion of the array 22 is illustrated with microneedles 10 protruding from a microneedle substrate surface 14. The microneedles 10 may be arranged in any desired pattern or distributed over the microneedle substrate surface 14 randomly. As shown, the microneedles 10 are arranged in uniformly spaced rows. In one embodiment, arrays of the present invention have a skin-facing surface area of more than about 0.1 cm² and less than about 20 cm², preferably more than about 0.5 cm² and less than about 5 cm². As shown, a portion of the substrate surface 16 of the patch 20 is non-patterned. In one embodiment the non-patterned surface has an area of more than about 1 percent and less than about 75 percent of the total area of the device surface that faces a skin surface of a patient. In one embodiment the non-patterned surface has an area of more than about 0.10 square inch (0.65 cm²) to less than about 1 square inch (6.5 cm²). In another embodiment (not shown), the microneedles are disposed over substantially the entire surface area of the array 22.

The microneedle devices useful in the various embodiments of the invention may comprise any of a variety of configurations, such as those described in the following patents and patent applications, the disclosures of which are herein incorporated by reference. One embodiment for the microneedle devices comprises the structures disclosed in United States Patent Application Publication No. 2003/0045837. The disclosed microstructures in the aforementioned patent application are in the form of microneedles having tapered structures that include at least one channel formed in the outside surface of each microneedle. The microneedles may have bases that are elongated in one direction. The channels in microneedles with elongated bases may extend from one of the ends of the elongated bases towards the tips of the microneedles. The channels formed along the sides of the microneedles may optionally be terminated short of the tips of the microneedles. The microneedle arrays may also include conduit structures formed on the surface of the substrate on which the microneedle array is located. The channels in the microneedles may be in fluid communication with the conduit structures. Another embodiment for the microneedle devices comprises the structures disclosed in co-pending U.S. patent application Ser. No. 10/621620 filed on Jul. 17, 2003 which describes microneedles having a truncated tapered shape and a controlled aspect ratio. Still another embodiment for the microneedle devices comprises the structures disclosed in U.S. Pat. No. 6,091,975 (Daddona, et al.) which describes blade-like microprotrusions for piercing the skin. Still another embodiment for the microneedle devices comprises the structures disclosed in U.S. Pat. No. 6,313,612 (Sherman, et al.) which describes tapered structures having a hollow central channel. Still another embodiment for the micro arrays comprises the structures disclosed in International Publication No. WO 00/74766 (Garstein, et al.) which describes hollow microneedles having at least one longitudinal blade at the top surface of tip of the microneedle.

Microneedle devices suitable for use in the present invention may be used to deliver drugs (including any pharmacological agent or agents) through the skin in a variation on transdermal delivery, or to the skin for intradermal or topical treatment, such as vaccination.

In one aspect, drugs that are of a large molecular weight may be delivered transdermally. Increasing molecular weight of a drug typically causes a decrease in unassisted transdermal delivery. Microneedle devices suitable for use in the present invention have utility for the delivery of large molecules that are ordinarily difficult to deliver by passive transdermal delivery. Examples of such large molecules include proteins, peptides, nucleotide sequences, monoclonal antibodies, DNA vaccines, polysaccharides, such as heparin, and antibiotics, such as ceftriaxone.

In another aspect, microneedle devices suitable for use in the present invention may have utility for enhancing or allowing transdermal delivery of small molecules that are otherwise difficult or impossible to deliver by passive transdermal delivery. Examples of such molecules include salt forms; ionic molecules, such as bisphosphonates, preferably sodium alendronate or pamedronate; and molecules with physicochemical properties that are not conducive to passive transdermal delivery.

In another aspect, microneedle devices suitable for use in the present invention may have utility for enhancing delivery of molecules to the skin, such as in dermatological treatments, vaccine delivery, or in enhancing immune response of vaccine adjuvants. In one aspect, the drug may be applied to the skin (e.g., in the form of a solution that is swabbed on the skin surface or as a cream that is rubbed into the skin surface) prior to applying the microneedle device.

Microneedle devices may be used for immediate delivery, that is where they are applied and immediately removed from the application site, or they may be left in place for an extended time, which may range from a few minutes to as long as 1 week. In one aspect, an extended time of delivery may from 1 to 30 minutes to allow for more complete delivery of a drug than can be obtained upon application and immediate removal. In another aspect, an extended time of delivery may be from 4 hours to 1 week to provide for a sustained release of drug.

The present invention has been described with reference to several embodiments thereof. The foregoing detailed description and examples have been provided for clarity of understanding only, and no unnecessary limitations are to be understood therefrom. It will be apparent to those skilled in the art that many changes can be made to the described embodiments without departing from the spirit and scope of the invention. Thus, the scope of the invention should not be limited to the exact details of the compositions and structures described herein, but rather by the language of the claims that follow.

1. An application device for applying a microneedle device to a skin surface comprising:
means for releasably retaining a microneedle array;
means for remotely detecting a distance between the applicator and a skin surface;
means for allowing triggering of the applicator in response to the detected distance; and
means for propelling the microneedle array into a skin surfaces

2. An application device for applying a microneedle device to a skin surface comprising:
a. means for releasably retaining a microneedle array;
b. a distance sensor capable of remotely sensing distance;
c. a triggering mechanism controlled by the distance sensor; and
d. means for propelling the microneedle array into a skill surface.

3. An application device according to claim 1 wherein the means for releasably retaining the microneedle array is selected from the group consisting of a repositionable adhesive, a hook and loop connection, a magnetic connection, a mechanical interference fit, and a snap-fit connection.

4. An application device according to claim 1 wherein the means for propelling the microneedle array into the skin surface employs an energy source selected from the group consisting of pressure, electricity, elastic bands, and magnets.

5. An application device according to claim 1 wherein the means for remotely detecting a distance between the applicator and a skin surface comprises a light source.

6. An application device according to claim 2 to wherein the distance sensor comprises a light source.

7 An application device according to claim 5 wherein the light source is a laser.

8. An application device according to wherein the means for remotely detecting a distance between the applicator and a skin surface comprises an ultrasonic sensor.

9. (canceled)

10. (canceled)

11. An application device according to claim 1 wherein the microneedle device comprises a patch having a backing, a microneedle array, and a pressure sensitive adhesive on a skin-facing surface of the patch.

12. A method of applying a microneedle device to a skin surface comprising:
a) providing an application device having a releasably retained microneedle device and an energy source suitable for propelling the microneedle array into the skin surface:
b) bringing the application device adjacent to but not contacting the skin surface;
c) sensing the distance between the skin surface and the application device; and
d) driving the microneedle device into the skin surface.

13. A method according to claim 12 wherein the microneedle device comprises a patch having a backing, a microneedle array, and a pressure sensitive adhesive on a skin-facing surface of the patch.

14. A method according to claim 13 wherein the only portion of the microneedle device that contacts the skin surface is the skin-facing portion of the patch.

15. An application device according to claim 2 wherein the means for releasably retaining the microneedle array, is selected from the group consisting of a repositionable adhesive, a hook and loop connection, a magnetic connection, a mechanical interference fit and a snap-fit connection.

16. An application device according to claim 2 wherein the means for prope1ling the microneedle array into the skin surface employs an energy source selected from the group consisting of pressure, electricity, elastic bands, and magnets.

17. An application device according to claim 2 wherein the light source is a laser.

18. An application device according to claim 2 wherein the microneedle device comprises a patch having a backing, a microneedle array, and a pressure sensitive adhesive on a skin-facing surface of the patch.
